(19) United States
(12) Patent Application Publication
(10) Pub. No.: US 2013/0103165 A1
(43) Pub. Date: Apr. 25, 2013

(54) BIOCOMPATIBLE EXTREMELY FINE TANTALUM FIBER SCAFFOLDING FOR BONE AND SOFT TISSUE PROSTHESIS

(71) Applicant: COMPOSITE MATERIALS TECHNOLOGY, INC., Shewsbury, MA (US)

(72) Inventor: James Wong, Shrewsbury, MA (US)

(73) Assignee: COMPOSITE MATERIALS TECHNOLOGY, INC., Shewsbury, MA (US)

(21) Appl. No.: 13/713,885

(22) Filed: Dec. 13, 2012

Related U.S. Application Data
(63) Continuation-in-part of application No. 12/961,209, filed on Dec. 6, 2010, now abandoned.

(51) Int. Cl. A61F 2/00

(52) U.S. Cl. CPC A61F 2/0063
USPC 623/23.74

(57) ABSTRACT

A tissue implant member for implanting in living tissue is provided. The implant is formed of a fibrous mat of tantalum filament having a diameter less than about 10 microns.

### CROSS REFERENCE TO RELATED APPLICATIONS

This application is a continuation-in-part of my co-pending U.S. application Ser. No. 12/961,209, filed Dec. 6, 2010, which application in turn claims priority from U.S. Provisional Application Ser. No. 61/266,911, filed Dec. 4, 2009, U.S. Provisional Application Ser. No. 61/295,063, filed Jan. 14, 2010 and U.S. Provisional Application Ser. No. 61/314,878 filed Mar. 17, 2010, the contents of which applications are incorporated herein, in their entirety, by reference.

### FIELD OF THE INVENTION

The present invention relates to the use of extremely fine tantalum fibers as a scaffolding agent for repair and regeneration of defected bone tissue and as a porous metal coating of solid body parts have replacement such as knee, hip joints as well as for soft tissue fibers such as nerve, tendons, ligaments, cartilage, and body organ parts, and will be described in connection with such utility, although other utilities are contemplated.

### BACKGROUND ART

There is a substantial body of art describing various materials and techniques for using biocompatible implants in the human body. Some of the more important needs are for hip joints, knee and spine reconstruction and shoulder joints. These implants are usually metallic since they are load bearing structures and require relatively high strengths. To insure proper fixation to the bone, a porous metal coating is applied to the implant surface, and is positioned such that it is in contact to the bone. This is to promote bone growth into and through the porous coating to insure a strong bond between the bone and the metallic implant. This coating requires a high degree of porosity, typically greater than 50% and as high as 70-80% with open connective pore sizes varying from 100 μ to 500 μ. These implants must have significant compression strength to resist loads that these joints can experience. The modulus must also be closely matched to that of the bone to avoid stress degradation of the adjoining tissue.

In addition to the use of tantalum fibers for bone growth, repair and attachment of the implant, it can also be used effectively as a scaffold for soft tissue growth and can provide either a permanent or temporary support to the damage tissue/organ until functionalities are restored. Regardless of whether it's soft or hard tissue repair/replacement, all biomaterial will exhibit specific interactions with cells that will lead to stereotyped responses. The ideal choice for any particular material and morphology will depend on various factors, such are osteoinduction, Osteoconduction, angiogenesis, growth rates of cells and degradation rate of the material in case of temporary scaffolds.

Tissue engineering is a multidisciplinary subject combining the principles of engineering, biology and chemistry to restore the functionality of damaged tissue/organ through repair or regeneration. The material used in tissue engineering or as a tissue scafford can either be naturally derived or synthetic. Further classification can be made based on the nature of application such as permanent or temporary. A temporary structure is expected to provide the necessary support and assist in cell/tissue growth until the tissue/cell regains original shape and strength. These types of scaffolds are useful especially in case of young patients where the growth rate of tissues are higher and the use of an artificial organ to store functionality is not desired. However, in the case of older patients, temporary scaffolds fail to meet the requirements in most cases. These include poor mechanical strength, mismatch between the growth rate of tissues and the degradation rate of said scaffold. Thus older patients need to have a stronger scaffold, which can either be permanent or have a very low degradation rate. Most of the work on scaffolding has been done on temporary scaffolds owing to the immediate advantages realized of the materials used and the ease of processing. Despite early success, tissue engineers have faced challenges in repairing/replacing tissues that serve predominantly biomechanical roles in the body. In fact, the properties of these tissues are critical to their proper function in vivo. In order for tissue engineers to effectively replace these load-bearing structures, they must address a number of significant questions on the interactions of engineered constructs with mechanical forces both in vivo and in vitro.

Once implanted in the body, engineered constructs of cells and matrices will be subjected to a complex biomechanical environment, consisting of time-varying changes in stresses, strains, fluid pressure, fluid flow and cellular deformation behavior. It is now well accepted that these various physical factors have the capability to influence the biological activity of normal tissues and therefore may plays an important role in the success or failure of engineered grafts. In this regard, it is important to characterize the diverse array of physical signals that engineered cells experience in vivo as well as their biological response to such potential stimuli. This information may provide an insight into the long-term capabilities of engineered constructs to maintain the proper cellular phenotype.

Significant advances have been made over the last four decades in the use of artificial bone implants. Various materials ranging of metallic, ceramic and polymeric materials have been used in artificial implants especially in the field of orthopedics. Stainless steel (surgical grade) was widely used in orthopedics and dentistry applications owing to its corrosion resistance. However later developments included the use of Co—Cr and Ti alloys owing to biocompatibilities issues and bio inertness. Currently Ti alloys and Co—Cr alloys are the most widely used in joint prostheses and other biomedical applications such as dentistry and cardio-vascular applications. Despite the advantages of materials such as Ti and Co—Cr and their alloys in terms of biocompatibility and bio inertness, reports indicated failure due to wear and wear assisted corrosion. Ceramics was a good alternative to metallic implants but they too had their limitation in their usage. One of the biggest disadvantages of using metals and ceramics in implants was the difference in modulus compared to the natural bone. (The modulus of articular cartilage varies from 0.001-0.1 GPa while that of hard bone varies from 7-30 GPa). Typical modulus values of most of the ceramic and metallic implants used lies above 70 GPa. This results in stress shielding effect on bones and tissues which otherwise is useful in keeping the tissue/bone functional. Moreover rejection by the host tissue especially when toxic ions in the alloy, such as Vanadium in Ti alloy, are eluted causes discomfort in patients necessitating revisional operations to be performed. Polymers have modulus within the range of 0.001-0.1 GPa and have been used in medicine for applications which range from artificial implants, i.e., acetabular cup, to drug delivery systems owing to the advantages of being chemically inert, biodegradability and possessing properties, which lies close to the cartilage properties. With the developments in the use of artificial implants there were growing concerns on the biocompatibility of the materials used for artificial implants and the immuno-rejection by the host cells. This led to the research on the repair and regeneration of damaged organs and tissues, which started in 1980 with use of autologuous (use of grafts from same species) skin grafts. Thereafter the field of tissue engineering has seen rapid developments from the use of synthetic materials to naturally derived material that includes use of autografts, allografts and xenografts for repair or regeneration of tissues.

Surface terrain or topography is one of the important factors governing cell adhesion and proliferation, and there have been many studies carried out in recent times to investigate the suitability of materials such as spider webs and cover slips, fish scales, plasma clots, and glass fibers. Silk fibers also have been used extensively in surgical applications such as for sutures and artificial blood vessels. Cell adhesion to materials is mediated by cell-surface receptors, interacting with cell adhesion proteins bound to the material surface. In aiming to promote receptor medicated cell adhesion the surface should mimic the extracellular matrix (ECM). ECM proteins, which are known to have the capacity to regulate such cell behaviors as adhesion, spreading, growth, and migration, have been studied extensively to enhance cell-material interactions for both in vivo and in vitro applications. However, the effects observed for a given protein have been found to vary substantially depending on the nature of the underlying substrate and the method of immobilization. In biomaterial research there is a strong interest in new materials, which combine the required mechanical properties with improved biocompatibility for bone implants and soft tissue repair and replacement.

The foregoing discussion of the prior art derives in large part from an article by Yarlagadda, et al. entitled Recent Advances and Current Developments in Tissue Scaffolding, published in Bio-Medical Materials and Engineering 15(3), pp. 159-177 (2005).

See also U.S. Pat. No. 5,030,233 to Ducheyne, who discloses a mesh sheet material for surgical implant formed of metal fibers having a fiber length of about 2 mm to 50 mm, and having a fiber diameter of about 20 to about 200 um. According to the '233 patent if the fiber length is more than about 50 mm, manufacturing becomes difficult. In particular, for fiber lengths in excess of about 50 mm, sieving the fibers becomes impractical if not impossible. If the diameter of the fibers is less than about 20 microns, it is difficult to maintain the average pore size of at least 150 μm needed to assure ingrowth of bony tissue. If the fiber diameter is greater than about 200 μm, the flexibility and deformability become insufficient.

See also U.S. Pat. No. 4,983,184 to Steinemann which describes the use of metallic fibers formed specially of titanium and titanium alloy, bundled together for forming a reinforcing material for soft tissue. More particularly, Steinemann teaches only titanium and titanium alloys for producing artificial soft tissue components and/or for reinforcing natural soft tissue components comprising elongate titanium or titanium alloy wires of diameter less than 20 micrometers, bundled together for use as an artificial soft tissue component and reinforcement for a soft tissue component in a human or animal.

According to Steinemann, only Ti and its alloys are specified. It is well established that pure tantalum metal has excellent bio-compatible properties and has for many years been used in the medical field. More importantly, a recent example is described in a paper by Bobyn, Stackpool, Hacking, Tanzer and Krygier in the Journal of Bone & Joint Surgery (Br), Vol. 81-B, No. 5, Sep., 1999, and in U.S. Pat. No. 5,282,861 to Kaplan which describe the use of porous tantalum bio material for use to promote bone growth and adhesion of the metallic implants, which material currently is marketed by the Zimmer Corp.

### SUMMARY OF THE INVENTION

I have found that fibrous mats formed of tantalum filament, of less than about 10 microns diameter, advantageously may be used as a scaffolding for promoting soft tissue growth such as for nerves, tendons and cartilage, and also as a porous coating to including hard body parts such as bone.

### BRIEF DESCRIPTION OF THE DRAWINGS

Further features and advantages of the present invention will be seen from the following detailed description taken in conjunction with the accompanying drawings, wherein:

FIG. 1 is a schematic block diagram of one alternative process of the present invention;

FIG. 2 is a simplified side elevational view showing casting of a sheet in accordance with the present invention; and

FIG. 3 is a side elevational view of a scaffolding implant in accordance with the present invention with fiber diameter of 1 micron at 5,000×mag.

### DETAILED DESCRIPTION

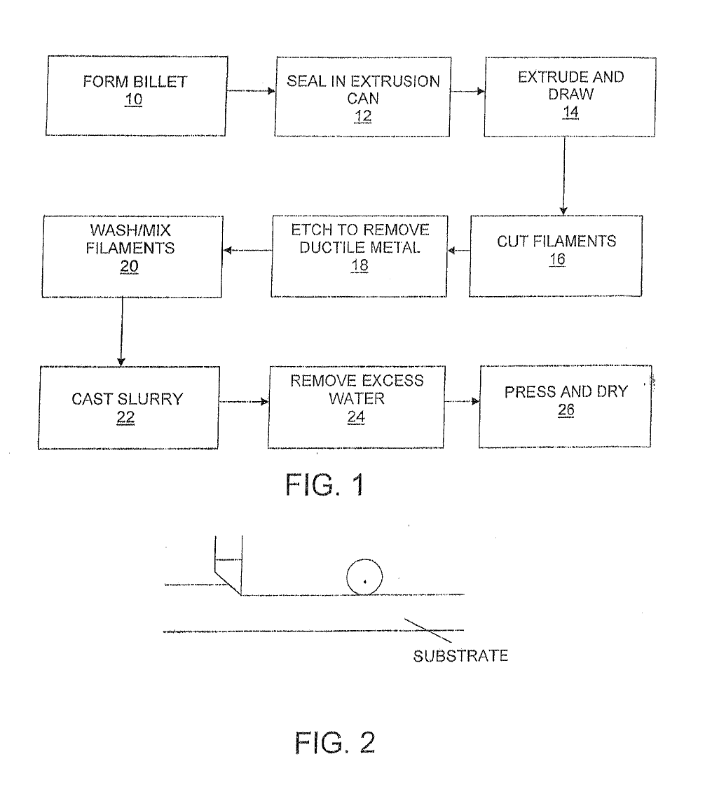

Referring to FIGS. 1 and 2, the process starts with the fabrication of valve metal filaments, such as tantalum, by combining shaped elements of tantalum with a ductile material, such as copper to form a billet at step 10. The billet is then sealed in an extrusion can in step 12, and extruded and drawn in step 14 following the teachings of my prior PCT applications Nos. PCT/US07/79249 and PCT/US08/86460, or my prior U.S. Pat. Nos. 7,480,978 and 7,146,709. The extruded and drawn filaments are then cut or chopped into short segments, typically 1/16^{th}-¼^{th} inch long at a chopping station 16. Preferably the cut filaments all have approximately the same length. Actually, the more uniform the filament, the better. The chopped filaments are then passed to an etching station 18 where the ductile metal is leached away using a suitable acid. For example, where copper is the ductile metal, the etchant may comprise nitric acid.

Etching in acid removes the copper from between the tantalum filaments. After etching, one is left with a plurality of short filaments of tantalum. The tantalum filaments are then washed in water, and the wash water is partially decanted to leave a slurry of tantalum filaments in water. The slurry of tantalum filaments in water is uniformly mixed and is then cast as a thin sheet using, for example, in FIG. 2 a “Doctor Blade” casting station 22. Excess water is removed, for example, by rolling at a rolling station 24. The resulting mat is then further compressed and dried at a drying station 26.

It was found that an aqueous slurry of chopped filaments will adhere together and was mechanically stable such that the fibers could easily be cast into a fibrous sheet, pressed and dried into a stable mat. Notwithstanding, as long as the filaments are not substantially greater than about 10 microns diameter, they easily adhere together. Filaments that are much larger than about 50 microns diameter, do not to form a stable mat. Thus, it is preferred that the filaments have a diameter of less than about 10 microns, and preferably less than about 5 microns, and preferably below 1 micron diameter. To ensure an even distribution of the filaments, and thus ensure production of a uniform mat, the slurry preferably is subjected to vigorous mixing by mechanical stirring and vibration. The porosity of the resulting tantalum fibrous sheet can be varied simply by pressing the mat further. Also, if desired, multiple layers may be stacked together to form thicker sheets.

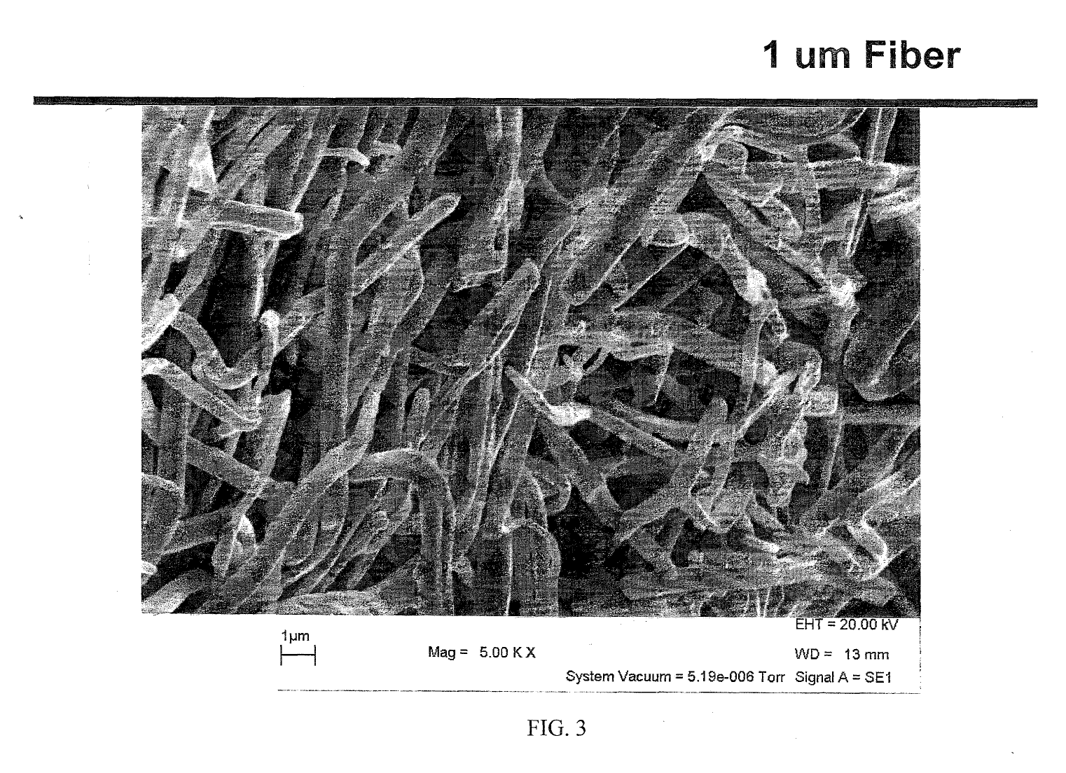

The resulting fibrous mat or sheet (FIG. 3) is flexible and has sufficient integrity so that it can be handled and shaped into an elongate scaffolding where it can then be used. The fibrous mat product made according to the present invention forms a porous surface of fibers having minimum spacings between fibers of approximately 100 to 500 microns which encourages healthy ingrowth of bone or soft tissue.

Numerous other arrangement by carding the fibers, meshes, braids and other fabric type arrangement can also be constructed.

The invention claimed is:

1. A tissue implant member for implanting in living tissue, comprising a fibrous mat of tantalum filaments in which the filaments have a diameter of less than about 10 microns.

2. The implant as in claim 1, wherein said tantalum filaments have a diameter of 0.5 to 10 microns.

3. The implant of claim 1, wherein the implant comprises a tissue scaffold for supporting soft tissue growth.

4. The implant of claim 3 wherein the tissue is selected from the group consisting of bone, nerve cells, tendons, cartilage and body organ parts.

5. A method for promoting soft tissue growth in a body comprising implanting in the body a tissue implant member as claimed in claim 1.

6. The method of claim 5, wherein the tissue is selected from the group consisting of bone, nerve cells, tendon, ligaments or cartilage and body organ parts.

7. A tissue implant member for implanting in living tissue comprising elongate threads or yarn of tantalum filaments in which the tantalum filaments have a diameter of less than about 10 microns.

8. The implant as in claim 7, wherein said tantalum filaments have a diameter of 0.5 to 10 microns.

9. The implant of claim 7, wherein the implant comprises a soft tissue scaffold for supporting tissue growth.

10. The implant of claim 7, wherein the tissue is selected from the group consisting of bone, nerve cells, tendon, ligaments, cartilage and body organ parts.

11. A method for promoting soft tissue growth in a body comprising implanting in the body a tissue implant member as claimed in claim 7.

12. The method of claim 11, wherein the tissue is selected from the group consisting of bone, nerve cells, tendon, ligaments, cartilage and body organ parts.
